(19) United States
(12) Patent Application Publication
(10) Pub. No.: US 2022/0304674 A1
(43) Pub. Date: Sep. 29, 2022

(54) CUTTING DEVICES FOR SUTURING TOOLS AND RELATED SYSTEMS AND METHODS

(71) Applicant: INTUITIVE SURGICAL OPERATIONS, INC., Sunnyvale, CA (US)

(72) Inventors: Ronald G. LITKE, Sandy Hook, CT (US); John F. GOODMAN, Ann Arbor, MI (US); Justin KROM, Southington, CT (US); Jake A. LUCKMAN, Milford, CT (US); Nathan VENSKYTIS, Woodbridge, CT (US)

(73) Assignee: INTUITIVE SURGICAL OPERATIONS, INC., Sunnyvale, CA (US)

(21) Appl. No.: 17/705,985

(22) Filed: Mar. 28, 2022

Related U.S. Application Data
(60) Provisional application No. 63/167,278, filed on Mar. 29, 2021.

(51) Int. Cl. A61B 17/04

(52) U.S. Cl. CPC A61B 17/0467; A61B 17/0491; A61B 17/0483; A61B 17/0482

(57) ABSTRACT

A needle driver device comprises an arcuate-shaped distal end portion defining an aperture opening at a distal end of the needle driver device and a cutting element disposed an exterior surface portion of the needle driver device. The cutting element is configured to cut suturing material. Devices, systems, and methods relate to cutting suturing material.

### CROSS REFERENCE TO RELATED APPLICATIONS

This application claims priority to U.S. Application No. 63/167,728, filed Mar. 29, 2021, the entire contents of which is incorporated by reference in its entirety.

### TECHNICAL FIELD

Aspects of the present disclosure relate to devices, systems, and methods for cutting suturing material. For example, aspects of the present disclosure relate to devices including, but not limited to, devices configured to cut suturing material during remote surgical, diagnostic, therapeutic, and other medical procedures. Further aspects of the disclosure relate to methods of operating such devices.

### INTRODUCTION

Sutures are used in a variety of medical applications, such as closing ruptured or incised tissue, soft tissue attachment, attachment of grafts, etc. Additionally, sutures may have other medical and/or non-medical uses. Conventionally, suturing is accomplished by penetrating tissue with the sharpened tip of a suturing needle that has a thread of suturing material attached to the opposite blunt end of the needle. The needle is then pulled through the tissue, causing the attached thread of suturing material to follow the path of the needle. Typically, a knot is tied at the trailing end of the thread to anchor the first stitch. This action is performed repetitively with application of tension to the needle to pull a length of the thread through the tissue using subsequent stitches until the tissue is sutured as desired with one or more stitches. At the conclusion of a suturing procedure, any excess amount of suturing material may be trimmed from the amount remaining after the knot at the trailing end.

While the above-described suturing process can be performed manually, automated suturing systems have also been developed. For example, some systems include a needle driver device configured to draw suturing material through tissue segments, similar to the manual suturing procedure described above.

Some automated suturing procedures utilize an additional tool, such as graspers, to manipulate tissue and/or suturing material at the surgical site to assist the suturing procedure. In some cases, the grasping tool can be swapped for a cutting tool to trim the suturing material. However, swapping the grasping tool for a cutting tool can incur additional procedure time, and introduction of an additional tool is undesirable from a clinical standpoint. Further, particularly in situations in which multiple suture lines are being inserted in the same general surgical area, multiple tool swaps would be required over the course of the procedure, further adding to overall procedure time.

It is desirable when performing certain suturing procedures to provide needle driver devices that occupy a minimal amount of space relative to a size (e.g., gauge and/or radius) of the needle. Such needle-drive devices are useful in space-limited applications, such as in the case of minimally invasive medical procedures, for example laparoscopic surgery or computer-assisted surgery.

A need exists to provide needle driver devices with an overall relatively small working end. A need also exists to streamline workflow of a suturing procedure, including trimming excess suturing material at the conclusion of suture insertion and/or when the suturing procedures uses long lengths of suturing material with multiple tie-off points.

### SUMMARY

Embodiments of the present disclosure may solve one or more of the above-mentioned problems and/or may demonstrate one or more of the above-mentioned desirable features. Other features and/or advantages may become apparent from the description that follows.

In accordance with at least one aspect of the present disclosure, a needle driver device comprises an arcuate-shaped distal end portion defining an aperture opening at a distal end of the needle driver device and a cutting element disposed an exterior surface portion of the needle driver device. The cutting element is configured to cut suturing material.

In yet another aspect of the present disclosure, a method of using a needle driver device comprising a cutting element comprises inserting the needle driver device to a site to perform a suturing procedure. The needle driver device comprises a cutting element carried at an exterior housing portion. The method further comprises driving a needle carrying suturing material through tissue to perform the suturing procedure and cutting excess suturing material using the cutting element.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are for example and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings,

FIG. 1 is a schematic, side view of an embodiment of a needle driver comprising a cutting element according to some embodiments of the present disclosure.

FIG. 2 is a perspective view of a distal end portion of a needle driver device comprising a cutting element according to some embodiments of the present disclosure.

FIG. 3 is a side view of the distal end portion of the needle driver device shown in FIG. 2.

FIG. 4 is a distal end view of the distal end portion of the medical device shown in FIGS. 2 and 3.

FIG. 5 is a perspective view of a cutting element and cutting element retainer according to some embodiments of the present disclosure.

FIG. 6 is a perspective view of a distal end portion of a needle driver device configured to receive the cutting element and retainer of FIG. 5.

FIGS. 7A-7F are various perspective views of the distal end portion of the needle driver device according to the embodiment of FIG. 6 and a cutting element retainer installation/removal tool according to some embodiments of the present disclosure showing a workflow for installing the cutting element retainer on the needle driver device.

FIGS. 8A-8E are various perspective view of the distal end portion of the needle driver device according to the embodiment of FIG. 6 and the cutting element retainer installation/removal tool of FIGS. 7A-7F showing a workflow for removing the cutting element retainer from the medical device.

FIG. 9 is a perspective view of a distal end portion of a needle driver device comprising a cutting element according to some embodiments of the present disclosure.

FIGS. 10A and 10B are perspective views of the distal end portion of the needle driver device of FIG. 9 cutting suturing material.

FIG. 11 is a perspective view of a distal end portion of a needle driver device comprising a cutting element according to some embodiments of the present disclosure.

FIGS. 12A and 12B are perspective views of the distal end portion of the needle driver device of FIG. 11 cutting suturing material.

FIGS. 13-15 are perspective views of distal end portions of needle driver devices comprising cutting elements according to some embodiments of the present disclosure.

FIG. 16 is a perspective view of a manipulator system according to some embodiments of the disclosure.

FIG. 17 is a partial schematic view of an embodiment of a manipulator system having a manipulator arm with two instruments in an installed position according to some embodiments of the present disclosure.

### DETAILED DESCRIPTION

Automated suturing systems can have particular application in conjunction with minimally-invasive surgical procedures. Minimally-invasive surgical procedures involve the use of remotely-controlled surgical instruments including, for example, teleoperated surgical instruments (e.g., surgical instruments operated at least in part with computer assistance, such as instruments operated with robotic technology) as well as manually operated (e.g., laparoscopic, thoracoscopic) surgical instruments. During such procedures, a surgical instrument, which may extend through a cannula inserted into a patient's body, can be remotely manipulated to perform a procedure at a surgical site. For example, in a teleoperated surgical system, cannulas and surgical instruments can be mounted at manipulator arms of a patient side cart and can be remotely manipulated via teleoperation at a surgeon console.

Following insertion of sutures at a site of a medical or other procedure, such as surgery, excess suturing material may be trimmed from the inserted sutures. Often in order to do so, an additional tool, such as surgical shears, may be introduced to the surgical site, for example, together with or after withdrawal of a needle driver device for applying sutures. This approach of using an additional tool to trim the suturing material can be undesirable in that it requires introduction of an additional tool at the surgical site and can result in increased procedure times. This may be particularly true in applications using long lengths of suture with multiple tie-off points or with the need to cut multiple lengths of suturing material.

According to various embodiments of the present disclosure, an automated suturing device, such as a needle driver device or other suturing tool can include a cutting element which can be used to trim the suturing material during or at the conclusion of a suturing procedure. The cutting element can be positioned at or on an exterior surface portion (such as an exterior housing portion) of the end effector portion of the needle driver device, and may be positioned so that the cutting element is conveniently located to trim the suturing material without requiring the suturing device to be removed from the surgical workspace (e.g., without entirely removing the suturing device from the cannula or other access device). In some embodiments, the cutting element may optionally be positioned at a distal end portion of the end effector portion.

In various embodiments, the cutting element can be arranged, such as via its positioning and/or orientation, so as to avoid a cutting edge of cutting element inadvertently snagging or otherwise catching on tissue or material other than the desired suturing material. In some embodiments, a cutting edge of the cutting element is positioned facing generally in a proximal direction. Further, the cutting element can be positioned so that the cutting element is close to a longitudinal centerline of the end effector portion of the needle driver device. In some embodiments, the cutting edge can be provided with a shroud to help protect the cutting edge. These arrangements can ensure that the cutting edge does not cause any inadvertent contact to tissue and/or other material or surfaces, as the needle driver device is being used in a procedure, is handled by a user, and/or is inserted or removed through a cannula to or from a remote site of a suturing procedure.

In some embodiments, the cutting element can be integrated with the suturing device as a permanent part of the device. Alternatively, the cutting element can be part of a replaceable and removable structure fitted to the suturing device.

In some embodiments, the cutting element is a passive cutting mechanism fixed in position relative to the suturing device and configured to be used by tensioning suturing material against the cutting edge to cut the suturing material. Alternatively, in some embodiments, the cutting element is actively movable, and upon actuation, shears the suturing material. In embodiments with an actively movable cutting element, a cutting edge can be completely enclosed in a housing in a retracted position and extendable from the housing so as to be exposed to perform the cutting operation.

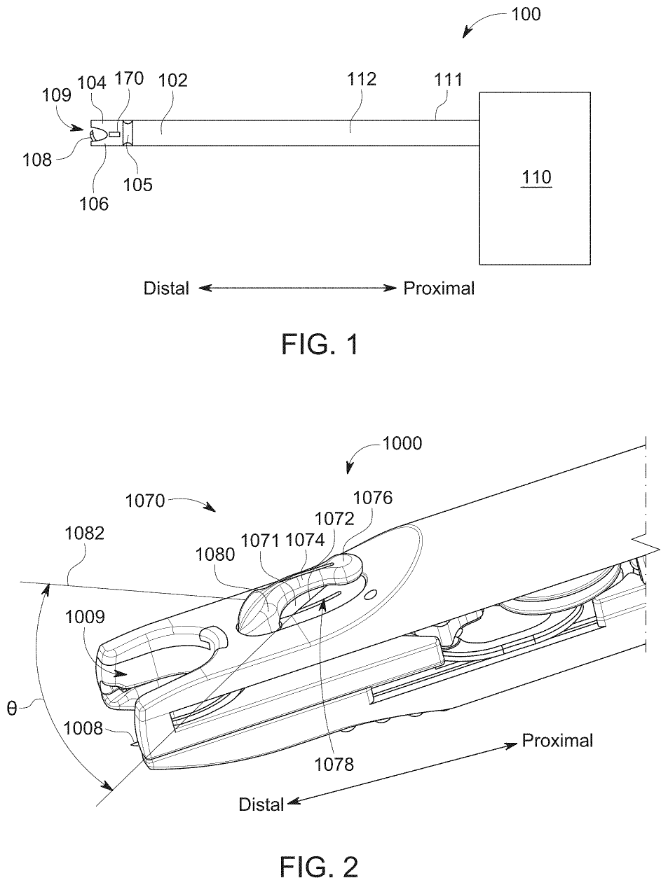

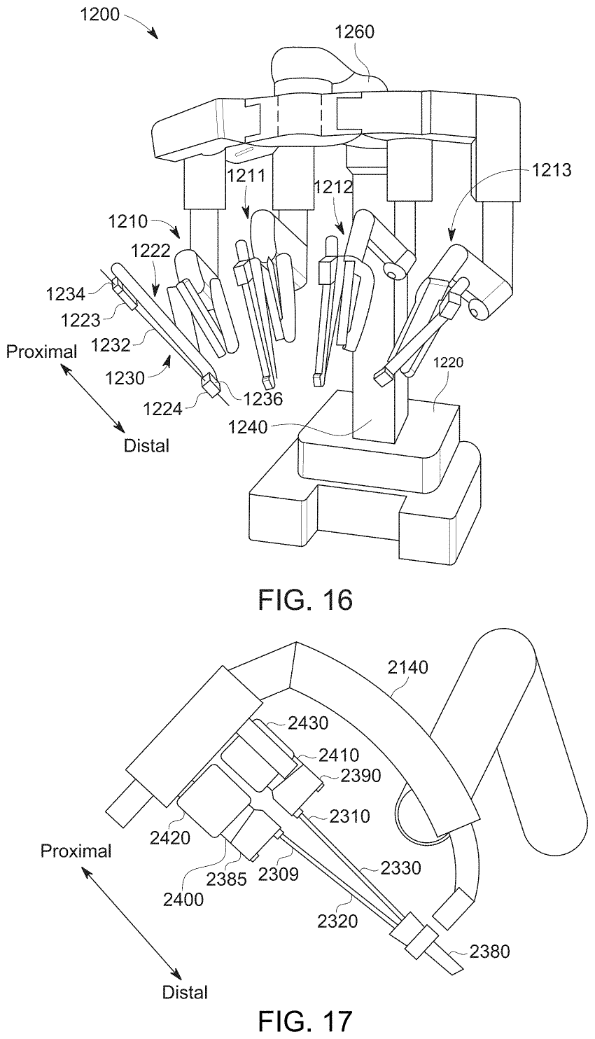

Referring now to FIG. 1, a schematic, side view of a needle driver device 100 according to some embodiments of the disclosure is shown. The needle driver device 100 includes an end effector 104, a shaft 112, and a transmission mechanism 110. The end effector 104 is located at a distal end portion 102 of the shaft 112. The end effector 104 comprises an arcuate-shaped distal end portion 106 (e.g., generally C-shaped and following a circular arc), which is configured to receive and house a curved needle 108 (e.g., also generally C-shaped, a sharpened tip portion of which is illustrated). In some embodiments, the needle 108 may be removable from the distal end portion 106 (e.g., for cleaning and sterilization). In some embodiments, the needle 108 has a curvature corresponding generally to the arc of the arcuate-shaped portion (e.g., generally C-shaped) of the end effector 104. The transmission mechanism 110 is coupled to a proximal end portion 111 of the shaft 112. The transmission mechanism 110 can be operably coupled with a computer-controlled (e.g., teleoperated) surgical manipulator system, such as the manipulator systems described in further detail below in connection with FIGS. 16 and 17, and/or the transmission mechanism 110 can be manually controlled with manually operated (e.g., handheld) actuators (not shown). The end effector 104 can optionally be coupled to the shaft 112 by a joint structure 105, such as a wrist, imparting one or more degrees of freedom to the end effector 104 relative to the shaft 112.

Drive inputs received at the transmission mechanism 110, whether through manual actuation or via a manipulator system, can actuate the end effector 104, such as by driving the needle 108 around a path defined partly by the arcuate-shaped distal end portion 106. The arcuate-shaped distal end portion 106 can face a distal direction of the end effector 104 and can define an opening or aperture 109, which may serve as a tissue gap for suturing tissue. Movement of the needle 108 across the aperture 109 of the arcuate-shaped distal end portion 106 can be used to, for example, suture tissue or other materials positioned within or adjacent the aperture 109 of the distal end portion 106. For example, the needle 108 may have a sharp or pointed leading portion that is configured to penetrate tissue or other material positioned in or adjacent the aperture 109. In some example embodiments, the arcuate-shaped distal end portion 106 includes an arcuate-shaped needle track, as discussed further below, exhibiting a radius of curvature similar to a radius of curvature of the needle 108, and the needle 108 rotates about a center of curvature of the arcuate-shaped track.

In the embodiment of FIG. 1, the needle driver device 100 includes a cutting element 170 located at the end effector 104 of the needle driver device 100. The cutting element 170 is configured to cut suture material. The cutting element 170 is located so as to be accessible during suturing of tissue or other material penetrated by the needle 108, and to cut off excess suturing material following suturing. The cutting element 170 may be located on an exterior of the needle driver device 100 and may be located on any suitable location of the exterior. In some embodiments, the cutting element 170 may be located proximal to the aperture 109. In other embodiments, the cutting element 170 may be located on the exterior of the needle driver device 100 at or near the aperture 109. In some embodiments, the cutting element 170 may be positioned adjacent the distal end portion 106 of the end effector 104. As discussed further herein, a preferred location of the cutting element may depend on a variety of factors, such as an overall size (e.g., diameter) of the needle driver device, an overall diameter of a needle of the needle driver device, and the resulting packaging constraints introduced by the relative size of the needle driver device and needle. The cutting element 170 can be a passive cutting element that passively cuts suturing material as discussed above or can be an active cutting element that shears the suturing material upon actuation of a movable cutting edge. Moreover, the cutting element can be integrated as part of the needle driver device 100 or can be removably attachable to the needle driver device 100.

Referring now to FIGS. 2-4, an end effector portion of a needle driver device 1000 is shown according to some embodiments. The needle driver device 1000 includes a reciprocating curved needle 1008 (the tip portion of which can be seen in FIG. 2) configured to move across an aperture 1009 to suture tissue or other materials positioned adjacent the aperture 1009. The needle driver device 1000 further includes a blade holder 1070 with a coupled blade 1071. The blade holder 1070 is positioned on an exterior of the end effector portion of the needle driver device 1000. The blade holder 1070 is located so as to be accessible during suturing of tissue or other material penetrated by the needle 1008, and to cut off excess suturing material following suturing. In the embodiment in FIG. 2, the blade holder 1070 is depicted as being proximal to the arcuate-shaped aperture 1009. However, the disclosure is not limited thereto or thereby, and the blade holder 1070 may be located on any suitable portion of the exterior of the needle driver device 1000. In some embodiments, the blade holder 1070 may be located on the exterior of the needle driver device 100 at or near the aperture 1009. In some embodiments, the blade holder 1070 may be positioned adjacent a distal end of the end effector portion.

As noted above, the location of the blade holder 1070 and blade 1071, or any of the cutting devices disclosed herein, can be chosen based on the overall size of the needle driver device (e.g., radial diameter of the device as required by a particular instrument/cannula system size, such as an 8 mm diameter system, 12 mm diameter system, 14 mm diameter system, or other size system) and the overall diameter of the needle relative to the overall size of the needle driver device. For example, the maximum depth of tissue penetration of which the needle driver device is capable can depend on an overall diameter of the needle (i.e., the diameter of a circular arc the needle traces during use). That is, a larger needle overall diameter can enable a greater depth of tissue penetration. It may be desired to maximize the overall diameter of the needle relative to the size of the needle driver device in order to enable a relatively greater depth of penetration capability for a given outside diameter of the needle driver device. As the needle overall diameter approaches the diameter of the needle driver device, there is less available room at the distal end of the needle driver device for other components, such as the blade holder 1070 and blade 1071, or any of the cutting element devices disclosed herein. As such, to accommodate a larger sized curved needle at the distal end of the device, the blade holder 1070 and blade 1071 may be positioned proximal to the aperture 1009.

Conversely, configuring a needle driver device with a needle having an overall diameter significantly less than the overall size (e.g., width or diameter) of the needle driver device could enable more components, e.g., including any of the cutting devices according to this disclosure, to be placed at or near the distal end of the needle driver device (e.g., adjacent the aperture 1009).

Accordingly, positioning of the cutting device may involve a compromise between overall needle diameter relative to the needle driver device and providing the cutting device near the distal end of the needle driver device to facilitate trimming of the suture closer to the work site. In other words, a relatively proximal location of the cutting device can facilitate configuration of the needle driver device for a needle diameter approaching a radial diameter of the needle driver device, thereby allowing for a relatively greater maximum possible tissue penetration depth for a given instrument size, such as an 8 mm instrument/cannula system, a 12 mm instrument/cannula system, 14 mm instrument/cannula system, or other size of system.

The blade 1071 can comprise a material suitable for forming a cutting edge 1072. For example, the blade can comprise materials, such as, for example, stainless steel, hardened steel, titanium, or other metals, alloys, or nonmetal materials (e.g., composite materials, ceramic, glass, etc.). The cutting edge 1072 can be arranged on the needle driver device 1000 to protect the cutting edge 1072 from undesired contact with other components and materials during use, during handled by a user, and/or during insertion and/or removal of the needle driver device 1000 for a procedure.

For example, in the embodiments of FIGS. 2-4, the cutting blade is arranged along a central axis of the end effector portion of the needle driver device 1000 extending between a proximal end and a distal end of the end effector portion (e.g., with a length of the cutting blade oriented approximately collinear with the central axis). The cutting edge 1072 of the blade 1071 is oriented (e.g., faces) towards a generally proximal direction. The blade holder 1070 includes a shroud 1074 partially covering the blade 1071. The shroud 1074 has a widened, rounded proximal portion 1076 that is configured to deflect components and tissue from entering a throat area 1078 proximate the blade 1071. Shoulders 1080 of the shroud 1074 located at a distal portion of the blade holder 1070 similarly deflect components and materials away from the blade 1071 to prevent such components and materials from inadvertently entering the throat area 1078. The proximal portion 1076 of the blade holder 1070 forms a free end of the shroud 1074. The shroud 1074 protrudes outwardly from the exterior of the needle driver device 1000 and extends in the proximal direction towards the free end of the proximal portion 1076.

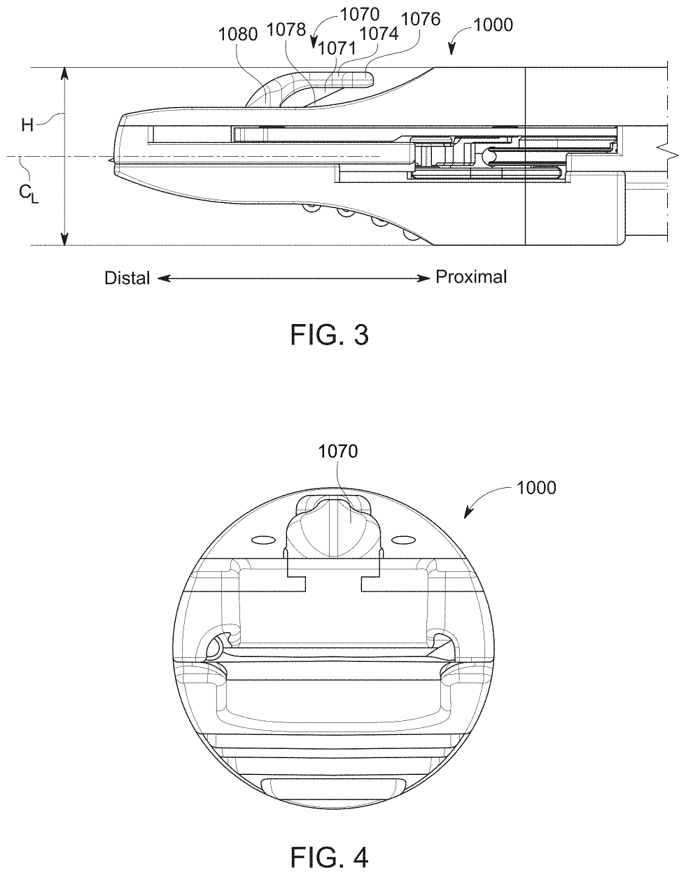

The overall profile of the shroud 1074 and blade 1071 can be configured such that they do not protrude beyond an overall exterior surface profile of the needle driver device 1000. For example, as shown in FIG. 3, which shows a partial side view of the needle driver device 1000 of FIG. 2, the shroud 1074 does not extend beyond the largest overall height H dimension of the distal end portion of needle driver device 1000. As is also apparent from FIG. 2, the cutting edge 1072 of the blade 1071 is oriented in a generally proximal direction and extends from the exterior of the distal end portion of the needle driver device 1000 at an acute angle. However, in alternate embodiments, the cutting edge 1072 of the blade 1071 may be oriented in a generally distal direction. In yet other alternative embodiments, the cutting edge 1072 of the blade 1071 may be oriented at an angle with respect to a central or longitudinal axis of the of the end effector portion of the needle driver device 1000. For example, the cutting the cutting edge 1072 may be oriented generally perpendicular to a longitudinal axis of the end effector portion of the needle driver device 1000. Referring now to FIG. 4, which shows an end view of the needle driver device 1000 of FIG. 2, the blade holder 1070 including the shroud 1074 is within the largest external surface profile of the needle driver device 1000.

Referring again to FIG. 2, to trim excess suturing material 1082, the needle driver device 1000 and/or the excess suturing material 1082 can be moved relative to one another (e.g., rotated, withdrawn, advanced, etc.) until the excess suturing material 1082 is positioned within the throat area 1078. Once the excess suturing material 1082 is in the desired position in the throat area, the needle driver device 1000 and/or the suturing material 1082 can be moved relative to one another (e.g., the needle driver device 1000 being moved in the proximal direction relative to the excess suturing material 1082 and/or the excess suturing material 1082 being moved in the distal direction) such that the excess suturing material is tensioned against and cut through by the cutting edge 1072.

The effectiveness of the cutting edge 1072 in cutting the excess suturing material 1082 can depend, at least in part, on the angle at which the excess suturing material 1082 contacts the cutting edge 1072. Stated another way, the excess suturing material 1082 must be pulled over the cutting edge 1072 at a relatively sharp acute angle to improve (e.g., maximize) the effectiveness of the cutting action. For example, in FIG. 2, the angle θ is equal to 90 degrees or less. To achieve this, the shoulders 1080 may be configured such that an angle formed between the cutting edge 1072 and an outermost profile of the shoulders 1080 matches the desired angle for the excess suturing material 1082. In other words, the shoulders 1080 may be configured and positioned so as to not interfere with the excess suturing material 1082 obtaining the desired angle θ for cutting effectiveness.

In the embodiment of FIGS. 2-4, the cutting element is permanently fixed to and integrated with the needle driver device 1000. However, in some applications, it may be desired to provide a cutting element that is removably attachable to a needle driver device. Such a removeable cutting element can facilitate replacement, e.g., of a dull cutting blade, without having to replace the entire needle drive device. In various embodiments, a removably attachable cutting element can be relatively simply installed and removed from the needle driver device without the use of separate attachment mechanisms, such as, for example, screws, clips, and the like. In this manner, field attachment and removal of the cutting element may be facilitated.

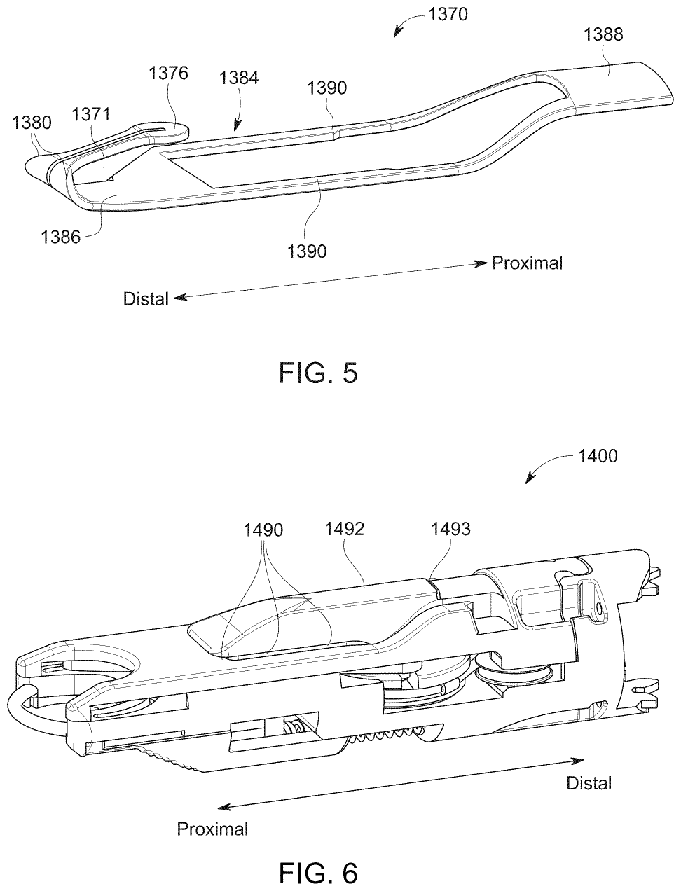

Referring now to FIGS. 5 and 6, a needle driver device that is configured to be fitted with a removably attachable cutting element is shown according to some embodiments. FIG. 5 shows a removably attachable cutting element 1370 that includes a blade 1371 and a blade retainer 1384 that can be installed on and removed from the needle driver device 1400 of FIG. 6. The blade 1371 can be attached to the blade retainer 1384 by, e.g., welding, crimping, or other attachment methods, or may be formed integrally with the blade retainer 1384, e.g., through molding or stamping. In some embodiments, the blade 1371 can be removably attached to the blade retainer 1384. The blade retainer 1384 comprises a blade mount portion 1386 and a retention tab 1388 coupled to one another by one or more rails 1390. When multiple rails 1390 are present, they may run parallel to one another and generally parallel to a longitudinal axis of the end effector of the needle driver device when coupled. The rails curve upward away from a plane in which the rails 1390 generally lie in the orientation of FIG. 5 to the location at which they couple to the retention tab 1388. The blade mount portion 1386 can be similar to the shroud 1074 discussed in connection with the embodiment of FIGS. 2-4, in that the blade mount portion 1386 has a rounded proximal end portion 1376 and shoulders 1380 extending outwardly over the blade 1371, all of which help to protect the blade 1371 from contacting tissue or other material or surfaces, as has been described above.

Referring now to FIG. 6, the needle driver device 1400 is shown without the removably attachable cutting element 1370 of FIG. 5 so as to better show various features that interact with the cutting element 1370 to couple the cutting element to the needle driver device 1400. The needle driver device 1400 has an undercut portion 1490 that receives a portion of the blade mount portion 1386 and the rails 1390. The needle driver device 1400 further has a ridge portion 1492, a proximal end of which has a proximally-facing registration surface 1493 configured to engage the retention tab 1388 of the blade retainer 1384 to hold the blade retainer 1384 and coupled cutting element 1370 on the needle driver device 1400.

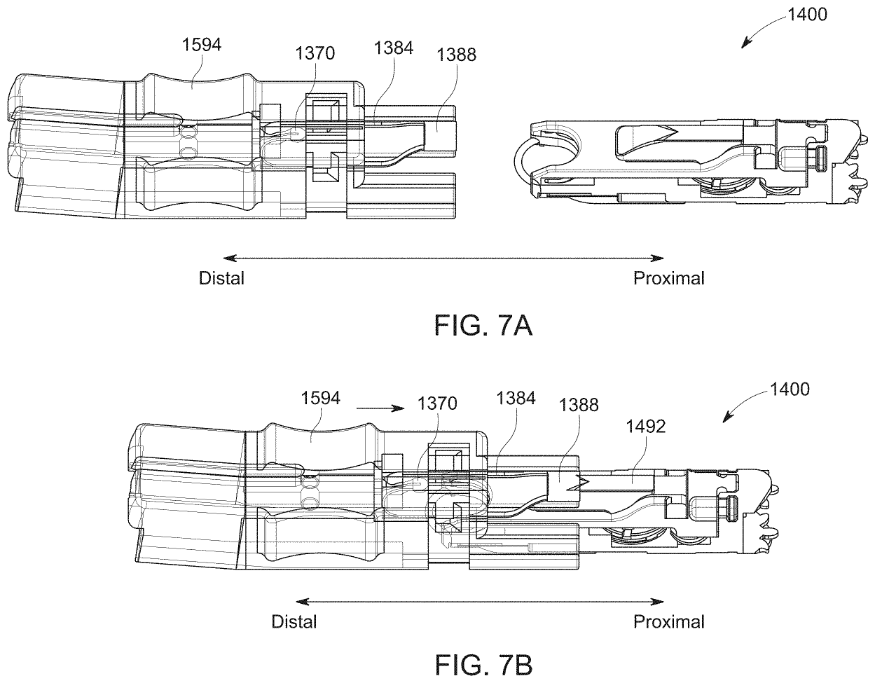

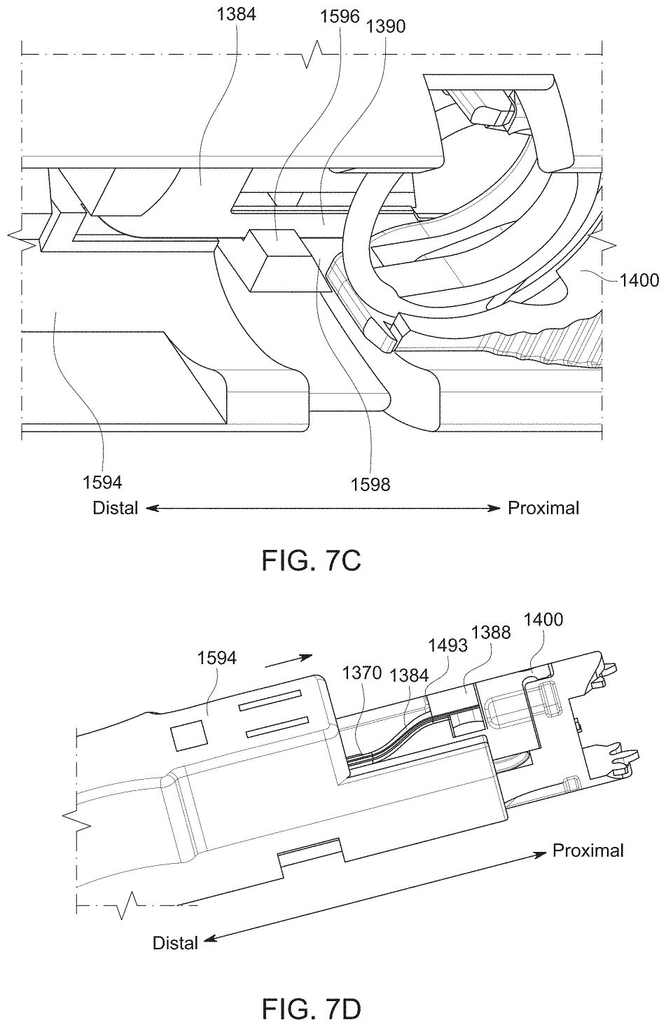

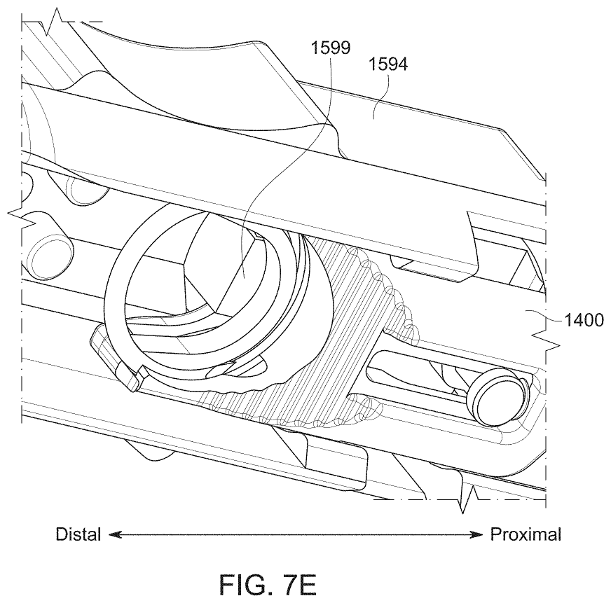

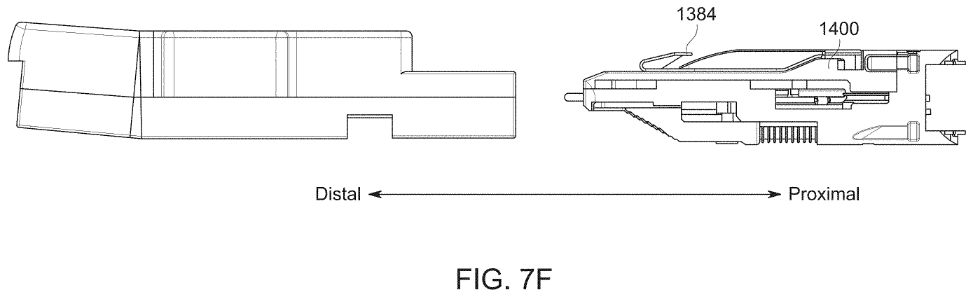

In some embodiments, a tool may be used to install and/or remove the blade retainer 1384 from the needle driver device 1400. For example, FIGS. 7A-7F show how an installation/removal tool 1594 can be used install the cutting element 1370. In FIG. 7A, the cutting element 1370 is held within the tool 1594, and the tool 1594 is advanced proximally toward and over the distal end portion of the needle driver device 1400, as shown in FIG. 7B. The retention tab 1388 of the blade retainer 1384 flexes slightly upward over the ridge portion 1492 of the needle driver device 1400 as the blade retainer 1384 moves proximally over the needle driver device 1400.

FIG. 7C shows a view of the needle driver device 1400 and tool 1594 from beneath (i.e., from the side of the needle driver device 1400 opposite the side shown in FIG. 7A). The tool 1594 includes lateral tabs 1596 that are engaged with the rails 1390 to releasably couple the blade retainer 1384 with the tool 1594 until installation of the blade retainer 1384 with the needle driver device 1400. As the tool 1594 is advanced proximally over the end effector portion of the needle driver device 1400, ramps 1598 on the lateral tabs 1596 bear against the needle driver device 1400 and the lateral tabs 1596 are deflected outward relative to the rails 1390 such that the rails engage the undercut portion 1490. Continued movement of the tool 1594 proximally over the needle driver device 1400 brings the retention tab 1388 of the blade retainer 1384 over the proximally-facing registration surface 1493, and the blade retainer 1384 snaps into place as shown in FIG. 7D, with the retention tab 1388 engaged against the proximally-facing registration surface 1493 as the lateral tabs 1596 release from the rails 1390.

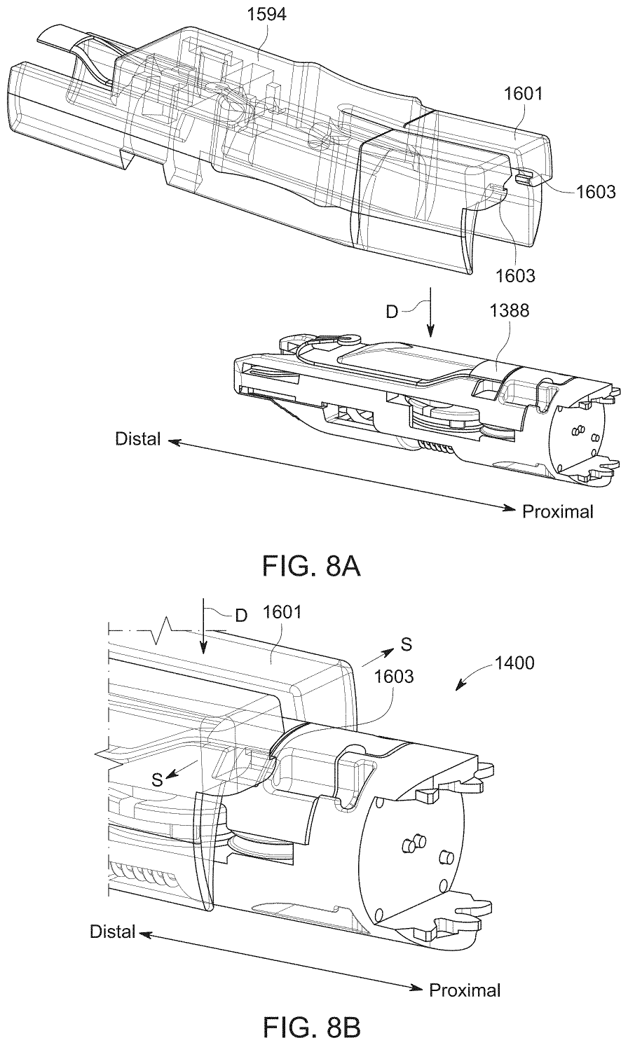

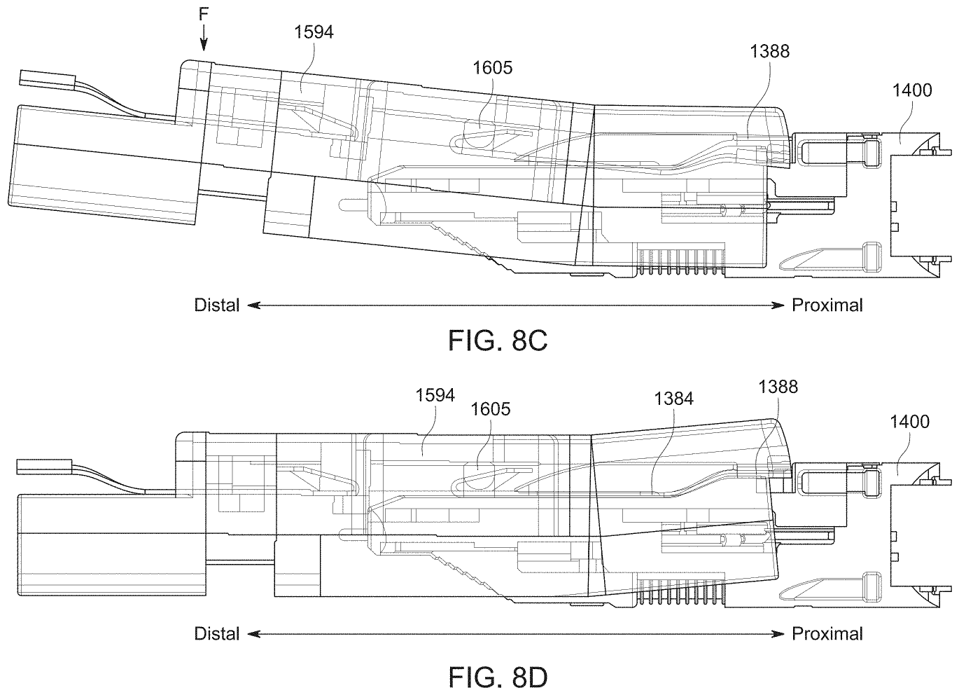

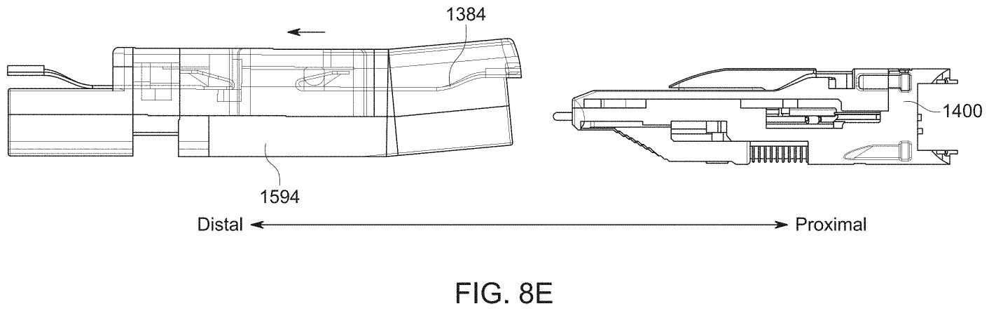

Referring now to FIG. 7E, which shows a view similar to FIG. 7C, as the blade retainer 1384 (FIG. 7D) seats within the undercut portion 1490 (FIG. 6) and the retention tab 1388 (FIG. 7D) engages the proximally-facing registration surface 1493 (FIG. 7D), a distal tab 1599 disengages the blade mount portion 1386 (FIG. 5) of the blade retainer 1384. The tool 1594 can then be pulled distally from the needle driver device 1400, leaving the cutting element 1370 installed on the needle driver device 1400 as shown in FIG. 8F. Use of the needle driver device 1400, and cutting element 1370, can then proceed as discussed above in connection with the embodiment of FIGS. 2-4, such as by manipulating the needle driver device 1400 as needed to cut excess suturing material with cutting element 1370.

To remove the cutting element 1370 from the needle driver device 1400, a removal end 1601 of the tool 1594 opposite the installation side can be advanced over the needle driver device 1400 in direction D, as shown in FIGS. 8A and 8B. The removal end 1601 of the tool 1594 includes proximal removal tabs 1603 (only one of which is visible in FIG. 8B) that, as shown in FIG. 8B, deflect around the retention tab 1388 as indicated by directional arrows S and engage the retention tab 1388 from underneath as the tool 1594 is pushed down over the needle driver device 1400 in direction D. The tool 1594 is rocked downward from the configuration shown in FIG. 8C to the configuration shown in FIG. 8D by a downward force F, which pulls the retention tab 1388 up and over the proximally-facing registration surface 1493 (FIG. 6), releasing the blade retainer 1384 from engagement with the needle driver device 1400. Distal removal tabs 1605 engage the blade mount portion 1386 of the blade retainer 1384, and the tool 1594 is pulled from the needle driver device 1400 as shown in FIG. 8E with the blade retainer 1384 held by proximal removal tabs 1603 and distal removal tabs 1605.

As noted above, the embodiments of FIGS. 2-4 and 5-8 are passive cutting elements in which cutting the suturing material is accomplished by relative motion between the needle driver device and the suturing material while the blade of the cutting element remains stationary relative to the needle drive device. In some embodiments of the present disclosure, active cutting elements are contemplated. Such active cutting elements are actuatable to move a cutting blade relative to the needle driver device in order access and cut suturing material. Such active cutting mechanisms can comprise shears, punches, anvils, and other various other actuatable cutting mechanisms. The active cutting mechanisms can be actuated by an actuator through the transmission mechanism (e.g., transmission mechanism 110) and a drive member that extends through a shaft of the instrument, or by other actuation components, such as other mechanical, electromechanical, hydraulic, pneumatic, or other actuation systems controlled manually or as part of a computer-assisted surgical system, such as the surgical systems disclosed in connection with FIGS. 16 and 17. The suturing material can be placed in the active cutting mechanism by, e.g., manipulating the position of the instrument of which the active cutting element forms a part, and/or by manipulating the suturing material directly, such as with a separate grasper instrument.

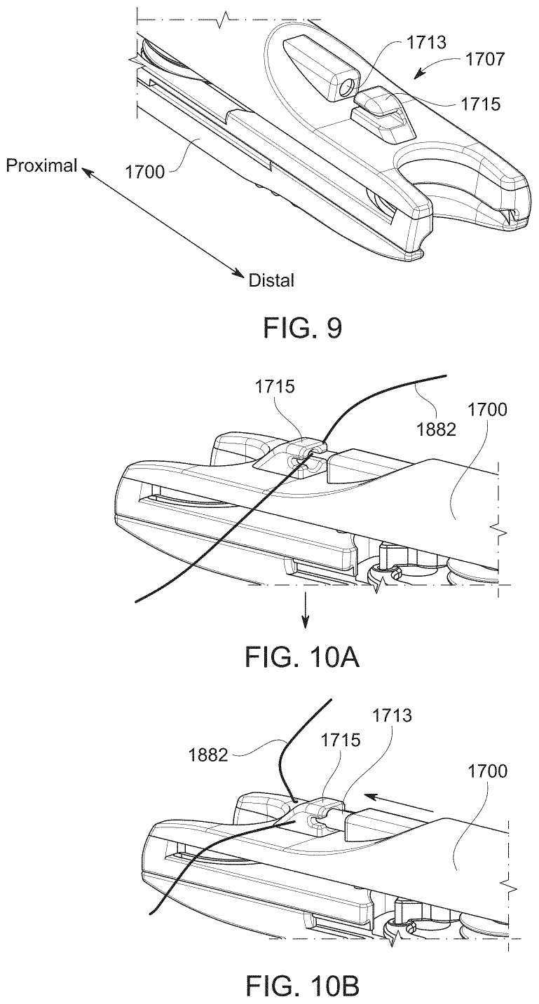

Referring now to FIG. 9, a needle driver device end effector portion 1700 with an active cutting element 1707 is shown according to some embodiments. The active cutting element 1707 is positioned at the needle driver device end effector portion 1700 in a location similar to that described above with reference to the embodiment of FIGS. 2 and 3. The active cutting element 1707 includes a movable element (e.g., pin) 1713 and an anvil 1715. The anvil 1715 is stationary with respect to the housing of the needle driver device 1700 and the movable element 1713 is actuatable to move relative to the needle drive device housing. The movable element 1713 and/or the anvil 1715 can comprise a hardened material such as stainless steel or may comprise another metal or nonmetal material.

Referring now to FIGS. 10A and 10B, to cut excess suturing material 1882, the needle driver device end effector portion 1700 and/or the suturing material is manipulated until the excess suturing material 1882 is brought against the proximal-facing side of the anvil 1715, as shown in FIG. 10A. The movable element 1713 is actuated and moves distally to the anvil 1715 to reach an extended position as shown in FIG. 10B. The excess suturing material 1882 is sheared between the moveable element 1713 and the anvil 1715, as shown in FIG. 10B. One or both of the movable element 1713 and the anvil 1715 may include cutting edges configured to shear the excess suturing material 1882 between the movable element 1713 and the anvil 1715.

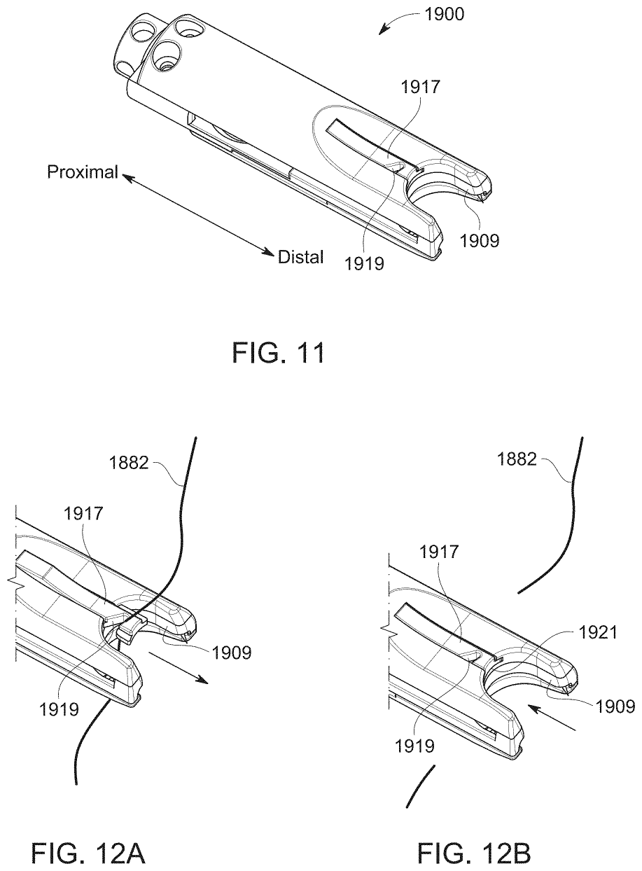

FIGS. 11, 12A, and 12B show another example embodiment of an active cutting element. Referring to FIG. 11, a needle driver device 1900 includes a movable element 1917 (e.g., a translating plunger) comprising notch 1919 extending from a lateral side of the movable element 1917. In the view of FIG. 11, the movable element 1917 is in a retracted position. To trim excess suturing material 1882, the movable element 1917 is extended distally to the fully extended position shown in FIG. 12A, so that the notch 1919 is exposed within the arcuate-shaped aperture 1909 of the needle driver device 1900. The suturing material 1882 is placed within the notch 1919, e.g., by manipulating the needle driver device 1900 and/or the suturing material 1882. Once the suturing material 1882 is placed in the notch 1919, the movable element 1917 is retracted proximally, and the suturing material 1882 is cut between an edge of the movable element 1917 within the notch and a cutting edge 1921 located on the needle driver device 1900 adjacent the movable element 1917, as shown in FIG. 12B. Positioning the movable element 1917 within the aperture 1909 can facilitate trimming the excess suturing material because the excess suturing material 1882 may be located within the aperture 1909 due to the action of the needle driver device, and thus require little manipulation of the needle driver device to position the excess suturing material 1882 within the notch 1919. While in the embodiment of FIGS. 11, 12A, and 12B, the cutting edge 1921 is located within the aperture 1909 adjacent the movable element 1917, in other example embodiments, the cutting edge may alternatively be positioned on in the notch 1919 of the movable element 1917, or each location can include a separate cutting element.

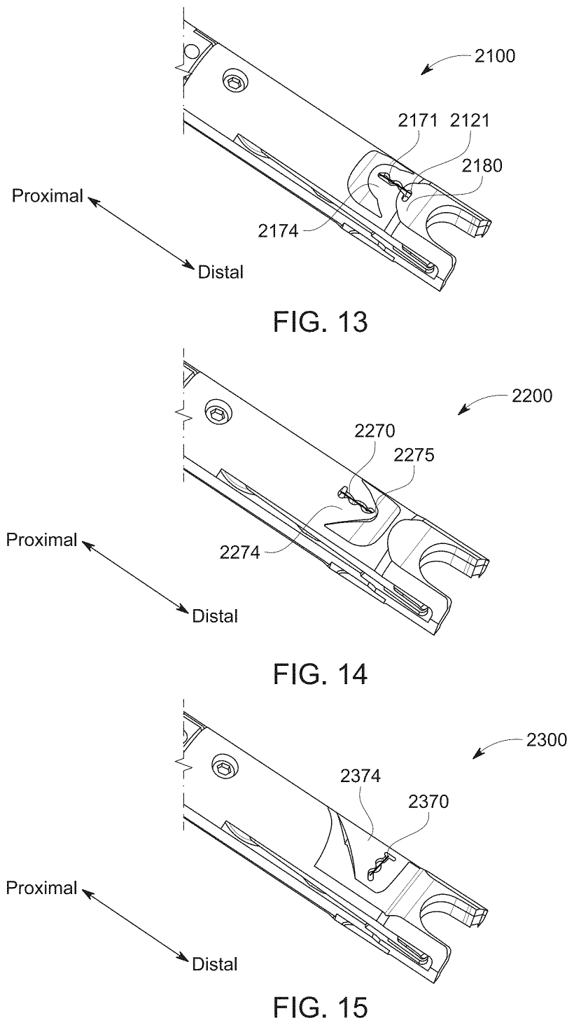

Referring now to FIGS. 13-15, additional configurations of passive cutting elements that are integrated with a needle drive device are shown. Referring to FIG. 13, a needle driver device 2100 includes a slot 2121 configured to hold a blade 2171 (e.g., similar to blade 1071 in FIG. 2). The general location and orientation of the blade 2171 is similar to that disclosed in connection with FIG. 2. However, in FIG. 13, a blade shroud 2174 comprises shoulders 2180 wider than the shoulders 1080 to further protect other components of the surgical system from contact with the blade 2171. Similar to the example embodiment of FIG. 2, in the embodiment of FIG. 13, excess suturing material must be positioned behind the blade shroud 2174, and the needle driver device 2100 moved proximally relative to the suturing material to cut the suturing material with the blade 2171.

FIG. 14 shows a needle driver device 2200 in which a cutting element is positioned facing generally distally. The needle driver device 2200 comprises a blade 2270 received in a blade shroud 2274, the blade shroud 2274 comprising a free end 2275 oriented in a distal direction. To trim excess suturing material, the excess suturing material is positioned underneath the blade shroud 2274 and adjacent the blade 2270, and the needle driver device 2200 is moved distally relative to the suturing material to cut the excess suturing material. The orientation of FIG. 14 can facilitate insertion and removal of the device 2200 through, e.g., a cannula, by preventing interference between the blade 2270 and/or blade shroud 2274 and other components, such as a cannula seal.

FIG. 15 shows another needle driver device 2300, in which the cutting element is positioned such that a cutting edge of a blade 2370 is positioned perpendicular to a longitudinal axis of the needle driver device 2300. A blade shroud 2374 extends in a direction perpendicular to the longitudinal axis of the needle driver device 2300. To trim excess suturing material, the excess suturing material is positioned behind the blade shroud 2374 and adjacent the blade 2370, and the needle driver device 2300 is rotated or moved laterally to cut the excess suturing material.

Embodiments of cutting elements according to the example embodiments of FIGS. 2-15 herein can be included with needle driver devices, e.g., any of the devices disclosed in connection with U.S. patent application Ser. No. 17/118,746 (filed Dec. 11, 2020) titled NEEDLE DRIVER DEVICES AND RELATED SYSTEMS AND METHODS, U.S. Provisional Patent Application No. [UNFILED—ATTORNEY DOCKET NO. P06395-US-PRV] titled DEVICES, SYSTEMS, AND METHODS FOR PERFORMING SUTURING PROCEDURES, or other devices. Additionally, cutting elements as disclosed herein in the embodiments of FIGS. 2-15 can be included on other medical devices, such as other needle driver devices including those configured to insert sutures using straight needles, or other medical devices such as manual suturing instruments.

Embodiments described herein may be used, for example, with remotely operated, computer-assisted systems (such, for example, teleoperated surgical systems) such as those described in, for example, U.S. Pat. No. 9,358,074 (filed May 31, 2013) to Schena et al., entitled “Multi-Port Surgical Robotic System Architecture”, U.S. Pat. No. 9,295,524 (filed May 31, 2013) to Schena et al., entitled “Redundant Axis and Degree of Freedom for Hardware-Constrained Remote Center Robotic Manipulator”, and U.S. Pat. No. 8,852,208 (filed Aug. 12, 2010) to Gomez et al., entitled “Surgical System Instrument Mounting”, each of which is hereby incorporated by reference in its entirety. Further, embodiments described herein may be used, for example, with a da Vinci® Surgical System, such as the da Vinci Si® Surgical System, da Vinci X® Surgical System, the da Vinci Xi® Surgical System, all with or without Single-Site® single orifice surgery technology, or the daVinci SP® Surgical System, all commercialized by Intuitive Surgical, Inc., of Sunnyvale, Calif.

The embodiments described herein are not limited to the surgical systems noted above, and various other teleoperated, computer-assisted surgical system configurations may be used with the embodiments described herein. Further, although various embodiments described herein are discussed in connection with a manipulating system of a teleoperated surgical system, the present disclosure is not limited to use with a teleoperated surgical system. Various embodiments described herein can optionally be used in conjunction with hand-held, manual instruments.

As discussed above, in accordance with various embodiments, surgical instruments of the present disclosure are configured for use in teleoperated, computer-assisted surgical systems employing robotic technology (sometimes referred to as robotic surgical systems). Referring now to FIG. 16, an embodiment of a manipulator system 1200 of a computer-assisted surgical system, to which surgical instruments are configured to be mounted for use, is shown. Such a surgical system may further include a user control system, such as a surgeon console (not shown) for receiving input from a user to control instruments coupled to the manipulator system 1200, as well as an auxiliary system, such as auxiliary systems associated with the da Vinci® systems noted above.

As shown in the embodiment of FIG. 16, a manipulator system 1200 includes a base 1220, a main column 1240, and a main boom 1260 connected to main column 1240. Manipulator system 1200 also includes a plurality of manipulator arms 1210, 1211, 1212, 1213, which are each connected to main boom 1260. Manipulator arms 1210, 1211, 1212, 1213 each include an instrument mount portion 1222 to which an instrument 1230 may be mounted, which is illustrated as being attached to manipulator arm 1210. While the manipulator system 1200 of FIG. 16 is shown and described having a main boom 1260 to which the plurality of manipulator arms are coupled and supported thereby, in other embodiments, the plurality of manipulator arms can be coupled and supported by other structures, such as an operating table, a ceiling, wall, or floor of an operating room, etc.

Instrument mount portion 1222 comprises a drive assembly 1223 and a cannula mount 1224, with a transmission mechanism 1234 (which may generally correspond to the transmission mechanism 110 discussed in connection with FIG. 1) of the instrument 1230 connecting with the drive assembly 1223, according to an embodiment. Cannula mount 1224 is configured to hold a cannula 1236 through which a shaft 1232 of instrument 1230 may extend to a surgery site during a surgical procedure. Drive assembly 1223 contains a variety of drive and other mechanisms that are controlled to respond to input commands at the surgeon console and transmit forces to the transmission mechanism 1234 to actuate the instrument 1230. Although the embodiment of FIG. 16 shows an instrument 1230 attached to only manipulator arm 1210 for ease of viewing, an instrument may be attached to any and each of manipulator arms 1210, 1211, 1212, 1213.

Other configurations of surgical systems, such as surgical systems configured for single-port surgery, are also contemplated. For example, with reference now to FIG. 17, a portion of an embodiment of a manipulator arm 2140 of a manipulator system with two surgical instruments 2309, 2310 in an installed position is shown. The surgical instruments 2309, 2310 can generally correspond to instruments discussed above, such as needle driver device 100 disclosed in connection with FIG. 1. For example, the embodiments described herein may be used with a DA VINCI SP® Surgical System, commercialized by Intuitive Surgical, Inc. of Sunnyvale, Calif. The schematic illustration of FIG. 17 depicts only two surgical instruments for simplicity, but more than two surgical instruments may be mounted in an installed position at a manipulator system as those having ordinary skill in the art are familiar with. Each surgical instrument 2309, 2310 includes a shaft 2320, 2330 that at a distal end has a moveable end effector or an endoscope, camera, or other sensing device, and may or may not include a wrist mechanism (not shown) to control the movement of the distal end.

In the embodiment of FIG. 17, the distal end portions of the surgical instruments 2309, 2310 are received through a single port structure 2380 to be introduced into the patient. As shown, the port structure includes a cannula and an instrument entry guide inserted into the cannula. Individual instruments are inserted into the entry guide to reach a surgical site.

Other configurations of manipulator systems that can be used in conjunction with the present disclosure can use several individual manipulator arms. In addition, individual manipulator arms may include a single instrument or a plurality of instruments. Further, as discussed above, an instrument may be a surgical instrument with an end effector or may be a camera instrument or other sensing instrument utilized during a surgical procedure to provide information, (e.g., visualization, electrophysiological activity, pressure, fluid flow, and/or other sensed data) of a remote surgical site.

Transmission mechanisms 2385, 2390 (which may generally correspond to transmission mechanism 110 disclosed in connection with FIG. 1) are disposed at a proximal end of each shaft 2320, 2330 and connect through a sterile adaptor 2400, 2410 with drive assemblies 2420, 2430. Drive assemblies 2420, 2430 contain a variety of internal mechanisms (not shown) that are controlled by a controller (e.g., at a control cart of a surgical system) to respond to input commands at a surgeon side console of a surgical system to transmit forces to the transmission mechanisms 2385, 2390 to actuate surgical instruments 2309, 2310.

The embodiments described herein are not limited to the embodiments of FIG. 16 and FIG. 17, and various other teleoperated, computer-assisted surgical system configurations may be used with the embodiments described herein. The diameter or diameters of an instrument shaft and end effector are generally selected according to the size of the cannula with which the instrument will be used and depending on the surgical procedures being performed.

This description and the accompanying drawings that illustrate various embodiments should not be taken as limiting. Various mechanical, compositional, structural, electrical, and operational changes may be made without departing from the scope of this description and the invention as claimed, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to another embodiment, the element may nevertheless be claimed as included in the other embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as examples. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

What is claimed is:

1. A needle driver device, comprising:
an arcuate-shaped distal end portion defining an aperture opening at a distal end of the needle driver device; and
a cutting element disposed an exterior surface portion of the needle driver device, the cutting element being configured to cut suturing material.

2. The needle driver device of claim 1, wherein the cutting element comprises a blade.

3. The needle driver device of claim 2, wherein the blade is fixed in position relative to the arcuate-shaped distal end portion.

4. The needle driver device of claim 3, wherein the blade comprises a cutting edge oriented in a generally proximal direction.

5. The needle driver device of claim 3, wherein the blade comprises a cutting edge oriented in a generally distal direction.

6. The needle driver device of claim 3, wherein the blade comprises a cutting edge oriented generally perpendicular to a longitudinal axis of the needle driver device.

7. The needle driver device of claim 4, wherein the cutting edge forms an acute angle with a longitudinal axis of the needle driver device.

8. The needle driver device of claim 7, wherein the cutting element further comprises a shroud at least partially covering the blade.

9. The needle driver device of claim 1, wherein the cutting element is removably engageable with the needle drive device.

10. The needle driver device of claim 9, wherein the cutting element comprises a blade and a blade retainer configured to be removably engageable with the needle driver device.

11. The needle driver device of claim 10, wherein the blade retainer comprises a blade mount portion, a retention tab configured to interface with a portion of the needle driver device, and one or more rails connecting the blade mount portion and the retention tab.

12. The needle driver device of claim 11, further comprising a housing comprising an undercut portion configured to receive one or both of a portion of the blade mount portion and a portion of the one or more rails.

13. The needle driver device of claim 1, wherein the cutting element is actuatable.

14. The needle drive device of claim 13, wherein the cutting element comprises a blade moveable in response to actuation of the cutting element.

15. The needle driver device of claim 14, wherein the blade is a pin moveable from a retracted position within a housing to an extended position external the housing in response to actuation of the cutting element.

16. The needle driver device of claim 15, wherein the pin is received in an anvil in the extended position.

17. The needle driver device of claim 16, wherein:
the cutting element further comprises a translating plunger having a notch in a lateral side, and
the blade is located within the notch.

18. The needle driver device of claim 17, wherein:
the translating plunger is configured to move distally into the aperture; and
wherein on the condition that the translating plunger is in a fully extended position, the notch of the translating plunger is located within the aperture.

19. The needle driver device of claim 1, wherein the cutting element is located proximal to the arcuate-shaped distal end portion.

20. A method of using a needle driver device comprising a cutting element, the method comprising:
inserting the needle driver device to a site to perform a suturing procedure, wherein the needle driver device comprises a cutting element carried at an exterior housing portion;
driving a needle carrying suturing material through tissue to perform the suturing procedure; and
cutting excess suturing material using the cutting element.

21. The method of claim 20, wherein cutting the excess suturing material using the cutting element comprises actuating a movable cutting element.

22. The method of claim 20, wherein cutting the excess suturing material comprises manipulating the needle driver device to cut the excess suturing material using the cutting element.

23. The method of claim 20, wherein cutting excess suturing material comprises cutting the excess suturing material in a central region of an arcuate-shaped path around which the needle traverses during a suturing procedure.
